(19) United States
(12) Patent Application Publication
(10) Pub. No.: US 2002/0197300 A1
(43) Pub. Date: Dec. 26, 2002

(54) Drug delivery system for anti-glaucomatous medication

(76) Inventors: Clyde L. Schultz, Boston, MA (US); Janet M. Mint, Ponte Vedra, FL (US)

Correspondence Address: CLARK & ELBING LLP, 101 FEDERAL STREET, BOSTON, MA, 02110, US

(21) Appl. No.: 10/179,344

(22) Filed: Jun. 25, 2002

Related U.S. Application Data
(63) Continuation-in-part of application No. 09/507,437, filed on Feb. 19, 2000, now Pat. No. 6,410,045.
(60) Provisional application No. 60/121,019, filed on Feb. 22, 1999.

(51) Int. Cl. A61K 9/00

(52) U.S. Cl. 424/429

(57) ABSTRACT

The invention features polymeric hydrogel contact lenses containing an anti-glaucoma medication, such as a beta adrenergic receptor antagonist, e.g., timolol maleate, or an alpha adrenergic receptor agonist, e.g., brimonidine tartrate, and methods of fabrication and uses thereof. A medication is passively transferred into a contact lens by absorption from a dilute aqueous solution. Such treated lenses are contacted with the ocular fluid of an individual to treat glaucoma.

### CROSS-REFERENCE TO RELATED APPLICATIONS

This application is a continuation-in-part of U.S. Application Ser. No. 09/507,437, filed Feb. 19, 2000, now U.S. Pat. No. 6,410,045, which claims benefit of U.S. Provisional Application No. 60/121,019, filed Feb. 22, 1999, each of which is hereby incorporated by reference.

### BACKGROUND OF THE INVENTION

The invention relates to the fields of drug delivery systems and treatments for glaucoma.

Glaucoma is a progressive optic neuropathy characterized by a specific pattern of damage to the head of the optic nerve and visual field. The visual system in glaucoma is damaged by the death of nerve cells, which carry the visual impulse from the eye to the brain. Once a sufficient number of nerve cells are destroyed, blind spots develop, usually beginning in the peripheral field of vision. Eventually central vision is affected. Since no treatment exists to restore these damaged nerve cells, this visual loss is irreversible. Glaucoma cannot currently be cured but can be effectively managed by medical or surgical treatment.

The single most important risk factor known for the development and or progression of glaucomatous damage is elevated intraocular pressure (IOP). Average IOP ranges between 14-22 millimeters of mercury (mmHg). A pressure of 22 or greater is considered to be elevated. Persons with IOP of 22 or greater are monitored and receive treatment to lower their IOP. In some individuals with elevated IOP no ocular damage can be detected, nonetheless, they receive prophylactic treatment to restore IOP to the normal range.

Numerous ocular drug delivery systems have been developed to manage IOP, but the complex anatomy of the eye has limited their effectiveness. Medications introduced into the eye are quickly washed out of the pre corneal area by the rapid production of lacrimal fluid. Additionally, medication in the eye is poorly absorbed because of the low permeability of corneal tissue.

Currently, dosing with ophthalmic medications in the form of drops results in a pattern of brief overdose of the eye medication when the drop is initially instilled, followed by a relatively short period of therapeutic dosing, followed by an interval in which the medication level drops to a less than therapeutic value. It has been determined that the ocular side effects and the more serious systemic side effects of ophthalmic drugs are primarily related to this period of initial drug overdose.

Systemic side effects experienced by the users of beta-adrenergic blocking drugs such as timolol maleate have included cardiac arrhythmias, life threatening bronchospasm and stroke. Therefore the use of beta-adrenergic blocking agents to treat glaucoma in patients diagnosed with significant cardiac or pulmonary disease requires careful monitoring and is often precluded altogether.

Additionally, a problem in the field of glaucoma treatment is the development of resistance to the commonly used anti-glaucoma medications by patients who eventually require increasing doses of their current medications or the addition of new medications to control IOP.

Ointments, gels, and high viscosity eye drops have been used to provide a longer acting formulation for anti-glaucoma medication. But these delivery systems have caused significant blurring of vision and ocular discomfort in many of those patients who have tried them. Ocular inserts have also produced substantial discomfort and often fall out of the eye of their users, after which they cannot be used again.

Another concern in the area of glaucoma treatment is the issue of patient compliance with prescribed treatment programs. Often topical delivery systems involve complicated, repetitious dosing schedules and the use of gels or drops, which can be awkward and difficult to apply.

The use of polymeric hydrogels as contact lenses to dispense medications in the eye is known as disclosed in U.S. Pat. Nos. 4,617,299; 4,668,506, and 5,723,131, each of which is hereby incorporated by reference. It is further known to use polymeric hydrogel contact lenses to deliver anti-glaucomatous medications in combination with corticosteroid medications to reduce IOP as disclosed in U.S. Pat. No. 5,212,168. Polymeric hydrogel contact lenses have also been used as carriers of antibiotics, which are dispensed into the eye as disclosed in U.S. Pat. No. 5,723,131.

It is known to presoak soft contact lenses such as Soflens® manufactured by Bausch & Lomb, in pilocarpine hydrochloride. However, some studies have found that this lens medicament delivery system may be unsuitable for use because the lens releases 100% of pilocarpine hydrochloride in buffered saline and distilled water in merely 1.5 and 2.5 hours respectively as disclosed in U.S. Pat. No. 4,731,244. Furthermore, while it is known in the art to simply presoak contact lenses in drug solutions, these medications commonly contain preservatives, such as benzalkonium chloride, which have a greater affinity for the hydrophilic contact lens material than do the aqueous drug solutions, with the result being the production of lenses with concentrated levels of preservative, which can be toxic to the corneal epithelium. (Bawa, R. Chapter 11, Ocular Inserts p. 231, citing Hillman, J. S. Br. J. Opthal., 58(7):674 (1975)).

In view of the many disadvantages of these prior medication delivery systems, there is a need for a new ophthalmic medication delivery system.

### SUMMARY OF THE INVENTION

The invention features polymeric hydrogel contact lenses containing an anti-glaucoma medication, such as a beta adrenergic receptor antagonist, e.g., timolol maleate, or an alpha adrenergic receptor agonist, e.g., brimonidine tartrate, and methods of fabrication and uses thereof. A medication is passively transferred into a contact lens by absorption from a dilute aqueous solution. The lenses of the invention are contacted with the ocular fluid of an individual, into which the medication is gradually released, to treat glaucoma.

Accordingly, in one aspect, the invention features a drug delivery system including a polymeric hydrogel contact lens that contains a beta adrenergic receptor antagonist, or a pharmaceutically acceptable salt thereof, at a concentration of between about 0.25% and 0.000005% by weight absorbed in the contact lens, wherein the beta adrenergic receptor antagonist is capable of being delivered into ocular fluid.

In another aspect, the invention features a drug delivery system including a polymeric hydrogel contact lens that contains an alpha adrenergic receptor agonist, or a pharmaceutically acceptable salt thereof, at a concentration of between about 0.2% and 0.000002% by weight absorbed in the contact lens, wherein the alpha adrenergic receptor agonist is capable of being delivered into ocular fluid.

The drug delivery systems of the invention may also include a combination of a beta adrenergic receptor antagonist and an alpha adrenergic receptor agonist.

The invention further features methods of fabricating a drug delivery system as described above. One method includes the steps of optionally washing a polymeric hydrogel contact lens in a saline solution; at least partially desiccating the lens; and contacting the washed and partially desiccated lens with a solution containing a beta adrenergic receptor antagonist, or a pharmaceutically acceptable salt thereof, at a concentration of about 0.25% to 0.000005% by weight. Another method includes the steps of optionally washing a polymeric hydrogel contact lens in a saline solution; at least partially desiccating the lens; and contacting the washed and partially desiccated lens with a solution containing an alpha adrenergic receptor agonist, or a pharmaceutically acceptable salt thereof, at a concentration of about 0.2% to 0.000002% by weight. In various embodiments, the lens is contacted with the solution for at least about 30 minutes. In other embodiments, the solution containing the anti-glaucoma drug has a pH of between about 7.0-7.4.

In other aspects, the invention features methods of controlling IOP in a mammal, e.g., a human, using the above-described drug delivery systems. One method includes the step of contacting a polymeric hydrogel contact lens with the ocular fluid of a mammal, wherein the contact lens contains a beta adrenergic receptor antagonist, or a pharmaceutically acceptable salt thereof, at a concentration of about 0.25% to 0.000005% by weight. Another method includes the step of contacting a polymeric hydrogel contact lens with the ocular fluid of a mammal, wherein the contact lens contains an alpha adrenergic receptor agonist, or a pharmaceutically acceptable salt thereof, at a concentration of 0.20% to 0.000002% by weight. In various embodiments, IOP is maintained at below about 22 mmHg. The drug may be released such that the concentration of the drug in the ocular fluid is approximately constant over a period of at least one day. In other embodiments, the above methods control the IOP for a period of at least 1 day, 2 days, 3 days, or 1 week.

Exemplary beta adrenergic receptor antagonists include timolol, levobunalol, carteolol, metipranolol, betaxolol, or a pharmaceutically acceptable salt thereof, or combinations thereof. Exemplary alpha adrenergic receptor agonists include brimonidine, apraclonidine, or a pharmaceutically acceptable salt thereof, or combinations thereof. In various embodiments of the above aspects, the pH of the ocular fluid is between about 7.0-7.4. A polymeric hydrogel contact lens used in the invention may have a water content of between about 10-90% by weight, e.g., between about 10 and 30%, 35%, 36%, 37%, or 37.9% or between about 90% and 60.1%, 61%, 62%, or 65%. Desirably, the polymeric hydrogel contact lens may contain a tetrapolymer of hydroxymethylmethacrylate, ethylene glycol, dimethylmethacrylate, and methacrylic acid. In certain embodiments, the anti-glaucoma drug is capable of being transferred into said ocular fluid under ambient or existing conditions. In other embodiments, the contact lens is capable of correcting vision.

By “treating” is meant the medical management of a patient with the intent that a prevention, cure, stabilization, or amelioration of the symptoms will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder.

By “pharmaceutically acceptable salt” is meant a non-toxic salt of a compound of the invention formed, e.g., from non-toxic inorganic or organic acids. Such non-toxic salts include, for example, those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Other pharmaceutically acceptable salts are known to those skilled in the art.

By “ambient conditions” is meant room temperature and pressure.

By “existing conditions” is meant in situ, as in the eye or other body system.

All percentages described in the specification are by weight, unless otherwise noted.

The drug delivery systems of the invention and methods of their use have several advantages over the prior art. The systems described herein effectively control elevated IOP by utilizing diluted doses of drugs, which are delivered to the ocular environment for a period of time longer than the dwell time of drop or gel formulations. The use of dilute doses decreases the probability that users will develop resistance to these drugs and would subsequently require increased doses or substitute medications to control IOP. Also the dilute concentrations used reduce the risk of systemic or ocular side effects from the drugs. This diminution in the risk of systemic side effects will enable the utilization of some drugs in some of those patients who would have been precluded from their use, e.g., because of existing cardiac or pulmonary conditions. The systems may also increase compliance by enabling the use of a single daily dose.

Other features and advantages of the invention will be apparent from the following description and claims.

### DETAILED DESCRIPTION OF THE INVENTION

We have invented a drug delivery system for the treatment of glaucoma. The system includes a polymeric hydrogel contact lens that contains a beta adrenergic receptor antagonist, e.g., timolol maleate, or an alpha adrenergic receptor agonist, e.g., brimonidine tartrate, at a low concentration. These systems are effective for lowering the IOP of individuals, while using 10% or less of the dosage typically required by conventional therapies.

Polymeric Hydrogel Contact Lens

This invention is principally directed at the passive transfer of an anti-glaucomatous medicament into a polymeric hydrogel contact lens and the subsequent delivery of this medicament into the ocular fluid of the eye.

The lenses of the invention are, for example, a polymeric hydrogel with a water content of about 10-90%, e.g., 38-60%, by weight. The polymer can be ionic, e.g., anionic, or nonionic. In one example, the composition of the polymer is a tetrapolymer of hydroxymethylmethacrylate, ethylene glycol, dimethylmethacrylate, and methacrylic acid. A monomer forming the hydrophilic polymer is, for example, the hydroxyester 2-hydroxyethyl methacrylate (HEMA). Exemplary hydrogel materials include etafilcon, polymacon, vifilcon, ocufilcon and omnifocon. The lenses may also be capable of correcting vision, for example, over a range of diopters of +8.0 to −8.0, including plano. The lenses may also have any base curve, e.g., from 8.0 to 9.0.

Anti-glaucomatous Medications

Any anti-glaucoma medication that can be delivered via the eye can be used with the drug delivery systems of the invention. The drug is desirably capable of being absorbed in a dilute form into the lens in an amount sufficient to allow a period of sustained delivery of this medication into the ocular fluid. These medications include beta adrenergic receptor antagonists, alpha adrenergic receptor agonists, miotics, and carbonic anyhydrase inhibitors. Other medications are known in the art. Desirably, the medication is a beta adrenergic receptor antagonist or an alpha adrenergic receptor agonist. Exemplary beta antagonists include timolol (e.g., hemihydrate or maleate), levobunalol, carteolol, metipranolol, and betaxolol. Exemplary alpha agonists include brimonidine (e.g., tartrate) and apraclonidine. Additional examples of anti-glaucoma medications include pilocarpine, epinephrine, dipivefrin, carbachol, acetazolamide, dorzolamide, brinzolamide, latanoprost, and bimatoprost. Combinations of anti-glaucoma drugs may also be used in the invention. Medications can be delivered to the eye in lower dosages than those typically used in gels or drops, because the drug delivery systems of the invention provide sustained release of the medications. The lower effective dosage may prevent or reduce side effects or the development of a tolerance. In certain embodiments, an anti-glaucoma drug is present in a lens at a concentration of less than about 10%, 1%, 0.1%, 0.01%, or 0.001% of a typical dosage (see, for example, Physicians' Desk Reference, 56^{th} ed., Medical Economics Company: Montvale, N.J. 2002). For beta antagonists, the concentration is, for example, at most about 0.25%, 0.05%, 0.025%, 0.005%, 0.0005%, or 0.00005% and at least about 0.000005%, 0.00005%, 0.0005%, 0.025%, or 0.05%. For alpha agonists, the concentration may be at most about 0.2%, 0.02%, 0.01%, 0.002%, 0.0002%, or 0.00002% and at least about 0.000002%, 0.00002%, 0.0002%, 0.002%, 0.01%, or 0.02%.

In addition to anti-glaucoma drugs, other medications may be included in the drug delivery systems of the invention. Examples of these other drugs include analgesics and antibiotics, cytokines, interleukins, anti-complement factors, or combinations thereof. The concentrations of any additional drugs may be reduced relative to their typical dosage by an amount similar to the reduction of the concentration of the anti-glaucoma drug employed.

Methods of Fabrication

In general, anti-glaucomatous medications are passively transferred to a contact lens by contacting the lens with a dilute aqueous solution of the drug. The drug is then passively transferred to the contact lens. Typically, the lens is washed in saline and desiccated, e.g., for at least about 15 minutes, 30 minutes, or 1 hour, prior to being contacted with the solution of drug at a pH of, e.g., about 7.0-7.4. In these embodiments, the desiccation removes greater than about 1%, 5%, 10%, 20%, 50%, or 75% or less than about 75%, 50%, 20%, 10%, 5%, or 1% of the water. The amount of desiccation may be used to control the amount of drug absorbed into the contact lens. The lens may be contacted with a solution of drug by immersion or spraying for a specific period of time, e.g., less than about 3 hours, 2 hours, 1 hour, 45 minutes, 30 minutes, or 15 minutes or greater than about 5 minutes, 10 minutes, 15 minutes, 30 minutes, or 1 hour. In other embodiments, the lens may be stored in an aqueous solution of a drug for an extended period of time, e.g., 6 hours, 12 hours, 24 hours, or longer. In certain embodiments, the drug is transferred to a contact lens from a non-aqueous solvent, e.g., dimethyl sulfoxide, which may be at least partially removed and replaced with an aqueous solution prior to use in a patient. Desirably, the solutions of drugs contain little or no preservatives, e.g., benzalkonium chloride, which may be toxic to the ocular tissue.

When employing desiccated lenses, the transfer of drug occurs at least in part by rehydrating the contact lens. Diffusion of the drug into the water in the lens may also occur. Typically, the concentration of drug transferred to the hydrogel is substantially lower than the solution with which the lens is contacted. For example, the concentration of drug in the lens is at least 2×, 5×, or 10× less than that of the soaking solution. The water content and type of lens, time and conditions, e.g., temperature, of soaking, composition of the soaking solution (e.g., ionic strength and pH), and type of drug employed may also influence the concentration of drug in the drug delivery system. The water content of the lens also helps to determine the total amount of drug present. Thus, the water content of a lens represents another variable by which to control the amount of drug delivered to the eye. The production of a lens containing a specified amount of a drug can be accomplished by routine experimentation by one skilled in the art.

Treatment Methods

To treat glaucoma, the lenses of the invention are contacted with the ocular fluid of an individual. The time period over which the lenses are worn may depend on the level of treatment desired or the amount of drug in the lens. Typically, the lenses will be worn for at least about 30 minutes, 1 hours, 2 hours, 3 hours, 6 hours, 12 hours, or longer. Once a lens has been worn, it may be cleaned and reused, for example, after additional drug has been passively transferred to the lens to replace that transferred to the eye.

The methods of treatment described herein are capable of delivering a drug to the ocular environment of a patient for a period of time longer than the dwell time achievable by gels or drops. In addition, the drug delivery system of the invention can be administered once daily. The convenience and simplicity of this system would in many cases enhance patient compliance with anti-glaucomatous therapy.

A further understanding of the invention may be obtained from the following non-limiting examples.

Patients, previously diagnosed with elevated IOP, were used as subjects in clinical tests conducted to determine whether or not hydrogel lenses containing passively transferred dilute concentrations of timolol maleate or brimonidine tartrate could effectively control increased IOP.

### EXAMPLE 1

A hydrogel contact lens for the right eye was prepared by washing an etafilcon A lens in a saline solution and then drying the lens briefly. This partially desiccated lens was then placed in an aqueous solution of brimonidine tartrate at a concentration 0.02% or 0.2 mg of brimonidine tartrate/ml, at a pH of 7.0-7.4 for 3 hours. This prepared lens was then tested in a patient who had previously been using one drop of a brimonidine tartrate 0.2% solution in his right eye every twelve hours which had maintained his IOP below 20 mmHg. The IOP in this patient's left eye was normal and did not require treatment. After a 4-day washout period in which all ocular medications were discontinued, the patient's IOP rose above 22 mmHg in the right eye. The patient then wore the lens treated with the brimonidine tartrate in his right eye for 30 minutes once a day. Within 24 hours the IOP in this patient's right eye had dropped to below 20 mmHg. No signs of ocular toxicity were noted upon subsequent slit lamp examination.

### EXAMPLE 2

The lenses were tested in a patient who had a history of elevated IOP in both eyes and who had been treated with one drop of 0.25% timolol maleate ophthalmic solution in both eyes every 12 hours, which controlled her IOP for approximately 2 years. Eventually her IOP rose, and it was necessary to change her anti-glaucoma medication to one drop every twelve hours of 0.25% timolol maleate ophthalmic gel forming solution. Provision of this medication in a gel carrier increases the time in which the medication remains in the ocular environment and should improve the efficacy of the drug. Following this treatment approach the patient's IOP remained controlled for the next 5 years. The patient then volunteered to test etafilcon A contact lenses which were prepared for use by washing the contact lenses in a saline solution and then drying the lenses briefly. These partially desiccated lenses were then placed in an aqueous solution of diluted timolol maleate at a concentration of 0.05% or 0.68 mg timolol maleate/ml for 3 hours. After this soaking period in which timolol maleate was passively transferred to the lenses, the lenses were subsequently worn by the patient for 30 minutes once a day. During the time this patient followed this regimen her IOP was maintained at less than 20 mmHg. Slit lamp examination after this treatment revealed no signs of ocular toxicity.

Subsequently the patient's elevated IOP was effectively managed with the administration of daily timolol maleate ophthalmic gel forming solution at one fifth of her previous dosage of this medication.

### EXAMPLE 3

The medicated lenses of this invention were tested in a patient who had a history of elevated IOP and had been controlled with a daily dose of 0.5% timolol maleate ophthalmic gel forming solution in each eye for two years. However, after this two year period the patient's IOP was no longer properly controlled by this medication regimen. The patient began a four-day washout period during which all anti-glaucoma medication was discontinued. The patient's IOP remained above 20 mmHg during this interval. After this period the patient then tested a set of etafilcon A hydrogel lenses which were prepared by washing them in a saline solution and then drying them briefly. These partially desiccated lenses were then soaked in a dilute, aqueous solution of timolol maleate 0.05% by weight or 0.68 mg timolol maleate/ml for 3 hours. Timolol maleate at this concentration was passively transferred to the lenses, which were then worn by the patient for 30 minutes once a day. The patient's IOP was controlled at a level of less than 20 mmHg for at least 6 months. No signs of ocular toxicity were noted on subsequent slip lamp examination.

Subsequently the procedure of Example 3 was repeated with 4 different types of polymeric hydrogel lenses, differing particularly in polymer type or water content. Each of the 4 differing types of polymeric hydrogel lenses, polymacon, vifilcon, ocufilcon and omnifocon, were capable of delivering an effective dose of timolol maleate, which had been passively transferred to the lens.

### EXAMPLE 4

Medicated lenses were prepared as follows. Vifilcon A lenses were soaked in a solution of 0.005% timolol maleate for approximately 3 hours. The patient of Example 3 then wore these vifilcon A lenses for 7 hours, after a wash-out period in which the patient's IOP was above 20 mmHg. The patient then removed the lenses, and IOP was controlled for the next 41 hours (48 from the time the lenses were applied to the eye).

### Other Embodiments

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A drug delivery system comprising a polymeric hydrogel contact lens comprising a beta adrenergic receptor antagonist, or a pharmaceutically acceptable salt thereof, at a concentration of between about 0.25% and 0.000005% by weight absorbed in said contact lens, wherein said beta adrenergic receptor antagonist is capable of being delivered into ocular fluid.

2. The drug delivery system of claim 1, wherein said ocular fluid has a pH of between about 7.0-7.4.

3. The drug delivery system of claim 1, wherein said polymeric hydrogel contact lens has a water content of between about 10-90% by weight.

4. The drug delivery system of claim 1, wherein said polymeric hydrogel contact lens comprises a tetrapolymer of hydroxymethylmethacrylate, ethylene glycol, dimethylmethacrylate, and methacrylic acid.

5. The drug delivery system of claim 1, wherein said beta adrenergic receptor antagonist is capable of being transferred into said ocular fluid under ambient conditions.

6. The drug delivery system of claim 1, wherein said beta adrenergic receptor antagonist is capable of being transferred into said ocular fluid under existing conditions.

7. The drug delivery system of claim 1, wherein said contact lens is capable of correcting vision.

8. The drug delivery system of claim 1, wherein said beta adrenergic receptor antagonist is selected from the group consisting of timolol, levobunalol, carteolol, metipranolol, betaxolol, or a pharmaceutically acceptable salt thereof, or combinations thereof.

9. The drug delivery system of claim 1, further comprising an alpha adrenergic receptor agonist.

10. A drug delivery system comprising a polymeric hydrogel contact lens comprising an alpha adrenergic receptor agonist, or a pharmaceutically acceptable salt thereof, at a concentration of between about 0.2% and 0.000002% by weight absorbed in said contact lens, wherein said alpha adrenergic receptor agonist is capable of being delivered into ocular fluid.

11. The drug delivery system of claim 10, wherein said ocular fluid has a pH of between about 7.0-7.4.

12. The drug delivery system of claim 10, wherein said polymeric hydrogel contact lens has a water content in the range of between about 10-90% by weight.

13. The drug delivery system of claim 10, wherein said polymeric hydrogel contact lens comprises a tetrapolymer of hydroxymethylmethacrylate, ethylene glycol, dimethylmethacrylate, and methacrylic acid.

14. The drug delivery system of claim 10, wherein said alpha adrenergic receptor agonist is capable of being transferred into said ocular fluid under ambient conditions.

15. The drug delivery system of claim 10, wherein said alpha adrenergic receptor agonist is capable of being transferred into said ocular fluid under existing conditions.

16. The drug delivery system of claim 10, wherein said contact lens is capable of correcting vision.

17. The drug delivery system of claim 10, wherein said alpha adrenergic receptor agonist is selected from the group consisting of brimonidine, apraclonidine, or a pharmaceutically acceptable salt thereof, or combinations thereof.

18. The drug delivery system of claim 10, further comprising a beta adrenergic receptor antagonist.

19. A method of fabricating a drug delivery system, said method comprising the steps of:
(a) optionally washing a polymeric hydrogel contact lens in a saline solution;
(b) at least partially desiccating said lens; and
(c) contacting the washed and partially desiccated lens of step (b) in a solution comprising a beta adrenergic receptor antagonist, or a pharmaceutically acceptable salt thereof, at a concentration of about 0.25% to 0.000005% by weight.

20. The method of claim 19, wherein said lens is contacted with said solution for at least about 30 minutes.

21. The method of claim 19, wherein said solution in step (c) has a pH of between about 7.0-7.4.

22. The method of claim 19, wherein said beta adrenergic receptor antagonist is selected from the group consisting of timolol, levobunalol, carteolol, metipranolol, betaxolol, or a pharmaceutically acceptable salt thereof, or combinations thereof.

23. A method of fabricating a drug delivery system, said method comprising the steps of:
(a) optionally washing a polymeric hydrogel contact lens in a saline solution;
(b) at least partially desiccating said lens; and
(c) contacting the washed and partially desiccated lens of step (b) in a solution comprising an alpha adrenergic receptor agonist, or a pharmaceutically acceptable salt thereof, at a concentration of about 0.2% to 0.000002% by weight.

24. The method of claim 23, wherein said lens is contacted with said solution for at least about 30 minutes.

25. The method of claim 23, wherein said solution in step (c) has a pH of between about 7.0-7.4.

26. The method of claim 23, wherein said alpha adrenergic receptor agonist is selected from the group consisting of brimonidine, apraclonidine, or a pharmaceutically acceptable salt thereof, or combinations thereof.

27. A method of controlling IOP in a mammal comprising contacting a polymeric hydrogel contact lens with the ocular fluid of said mammal, wherein said contact lens comprises a beta adrenergic receptor antagonist, or a pharmaceutically acceptable salt thereof, at a concentration of about 0.25% to 0.000005% by weight.

28. The method of claim 27, wherein said IOP is maintained at below about 22 mmHg.

29. The method of claim 27, wherein said beta adrenergic receptor antagonist is released such that the concentration of said antagonist in said ocular fluid is approximately constant over a period of at least one day.

30. The method of claim 27, wherein said IOP is controlled over a period of at least two days.

31. The method of claim 27, wherein said mammal is a human.

32. A method of controlling IOP in a mammal comprising contacting a polymeric hydrogel contact lens with the ocular fluid of said mammal, wherein said contact lens comprises an alpha adrenergic receptor agonist, or a pharmaceutically acceptable salt thereof, at a concentration of 0.20% to 0.000002% by weight.

33. The method of claim 32, wherein said IOP is maintained at below about 22 mmHg.

34. The method of claim 32, wherein said alpha adrenergic receptor agonist is released such that the concentration of said agonist in said ocular fluid is approximately constant over a period of at least one day.

35. The method of claim 32, wherein said IOP is controlled over a period of at least two days.

36. The method of claim 32, wherein said mammal is a human.
